(19) United States
(12) Patent Application Publication
(10) Pub. No.: US 2007/0259030 A1
(43) Pub. Date: Nov. 8, 2007

(54) Methods, systems and reagents for tendon and ligament therapy

(76) Inventor: Susan J. Drapeau, Cordova, TN (US)

Correspondence Address: FOX ROTHSCHILD LLP;PRINCETON PIKE CORPORATE CENTER, 997 LENOX DRIVE, BUILDING #3, LAWRENCEVILLE, NJ, 08648, US

(21) Appl. No.: 11/418,948

(22) Filed: May 5, 2006

(51) Int. Cl. A61K 38/55; A61K 9/127

(52) U.S. Cl. 424/450; 514/2

(57) ABSTRACT

A method for the use and delivery of tissue inhibitors of matrix metalloproteinase (TIMPs) to control the activity of the matrix metalloproteinase (MMPs) in the extracellular matrix (ECM) in order to treat and prevent extracellular matrix degradation in an injured tendon or ligament is presented. Accelerated breakdown of the extracellular matrix occurs in various pathological processes, including inflammation, chronic degenerative diseases and tumor invasion. Four members of the tissue inhibitor of metalloproteinase (TIMP) family have been characterized so far, designated as TIMP-1, TIMP-2, TIMP-3, and TIMP-4. Various introduction amounts, timing and combinations are capable of inhibiting the activities of all known matrix metalloproteinase (MMPs) and as such play a key role in maintaining the balance between (ECM) deposition and degradation in different physiological processes.

### FIELD OF THE INVENTION

The invention relates generally to the inhibition of extracellular matrix degradation in an injured tendon or ligament, and more particularly, to the use and delivery of Matrix Metalloproteinase (“MMP”) inhibitors to treat and prevent such extracellular matrix degradation in an injured tendon or ligament in order to promote natural healing and faster recovery.

### BACKGROUND OF THE INVENTION

Tendons, which connect muscle to bone, and ligaments, which connect bones to other bones, are both composed of bands of fibrous connective tissue. The cells of the fibrous connective tissue are mostly made up of fibroblasts—the irregular, branching cells that secrete strong fibrous proteins (such as collagens, reticular and elastic fibers, and glycoprotein's) as an extracellular matrix. The extracellular matrix can be defined in part as any material part of a tissue that is not part of any cell. So defined, the extracellular matrix (ECM) is the significant feature of the fibrous connective tissue.

The ECM's main component is various glycoprotein's. In most animals, the most abundant glycoprotein in the ECM is collagen. Collagen is tough and flexible and gives strength to the connective tissue. Indeed, the main element of the fibrous connective tissue are collagen (or collagenous) fibers. The ECM also contains many other components: proteins such as fibrin and elastin, minerals such as hydroxyapatite (the principal bone salt that provides the compressional strength of vertebrate bone), or fluids such as blood plasma or serum with secreted free flowing antigens. Given this diversity, it can serve any number of functions, such as providing support and anchorage for cells (which attach via focal adhesions), providing a way of separating the tissues, and regulating intercellular communication. The ECM functions, therefore, in a cell's dynamic behavior.

Injuries to the tendons and ligaments causes damage not only to the connective tissue, but to the extracellular matrix as well. Damage therefore to the ECM can interrupt cell behavior in the connective tissue and decrease and/or limit healing. After injury, continuing damage is caused by production of MMPs by the body. MMPs are enzymes that degrade all components of the ECM. This leads to an imbalance between the synthesis and degradation of the ECM, as the body tries to heal itself while the enzymes remodel the ECM. An overabundance of remodeling by MMPs cause damage to previously connected tissue which results in the formation of scar tissue. In addition, scar tissue adhesion to surrounding tissue can cause further pulling and/or stretching of the tendons or ligaments and resultant pain.

Currently, treatment of injury to tendons and ligaments includes some simple measures such as: avoiding activities that aggravate the problem; resting the injured area; icing the area the day of the injury; and taking over-the-counter anti-inflammatory medicines. However, these simple remedies do not always cure the injury and often more advanced treatments are needed. These treatments include: corticosteroid injections; physical therapy and even surgery. Corticosteroids (often called “steroids”) are often used because they work quickly to decrease the inflammation and pain. Physical therapy includes range of motion exercises and splinting (such as for the fingers, hands, and forearm). Surgery is only rarely needed for severe problems not responding to the other treatments.

In view of the foregoing, it can be appreciated that additional treatment measures are needed to treat and prevent extracellular matrix degradation for quicker and improved healing of tendons and ligaments.

### SUMMARY OF THE INVENTION

Accordingly, the present invention introduces the use and delivery of tissue inhibitors of matrix metalloproteinase (TIMPs) to control the activity of the MMPs in the ECM in order to treat and prevent extracellular matrix degradation in an injured tendon or ligament. TIMPs are a family of natural inhibitors and four members of this family have been so far characterized in a variety of species—designated as TIMP 1, TIMP 2, TIMP 3, and TIMP 4. Each of the four genus of inhibitors share a similar structural feature characterized by the presence of 12 cysteine residues involved in disulfide bonds and a similar function by their ability to form inhibitory complexes with MMPs. As such, introduction of each genus either individually, or in some combination, and either all at once or over some time progression results in prevention of ECM degradation.

Delivery of the TIMPs can be by a number of means, such as by a bolus injection, an application topically or an injection at the time of surgery. In addition, delivery may be by controlled release, oral, and/or in conjunction with an anti-inflammatory, a pain medication or a growth factor (such as LMP or BMP) to enhance tissue formation.

According to a first aspect of the present invention there is provided the use of TIMPs in the manufacture of a medicament for use in the treatment or prevention of fibrous connective tissue degradation.

According to a second aspect of the present invention there is provided a method of preventing or treating fibrous connective tissue degradation comprising administering to a subject in need of treatment a therapeutically effective amount of a TIMP. One embodiment of this aspect of the invention provides for a combined treatment with polyethylene glycols (PEG) and TIMPs to control the activity of the MMPs in the ECN in order to treat and prevent extracellular matrix degradation in an injured tendon or ligament.

The present invention, including its features and advantages, will become more apparent from the following detailed description with reference to the accompanying drawings.

### BRIEF DESCRIPTION OF THE DRAWINGS

FIG. 1 illustrates an injury (i.e., a tear or otherwise) to a tendon in the human body.

FIG. 2 illustrates a method for the utilization of an MMP inhibitor, according to an embodiment of the present invention.

### DETAILED DESCRIPTION

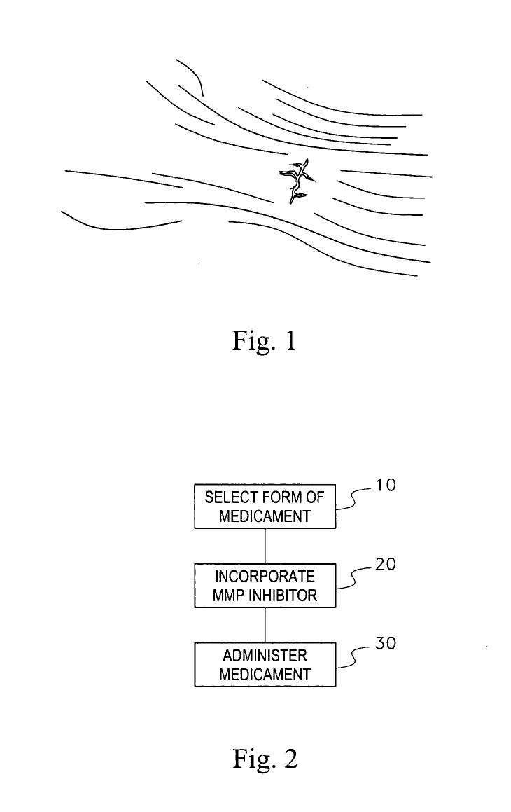

FIGS. 1 and 2 illustrate a common injury in a tendon or ligament, healing and recovery of which are assisted by the use and delivery of matrix metalloproteinase (MMP) inhibitors.

Referring now to FIG. 1, degradation and/or denaturization of fibrous connective tissues comprising extracellular matrix components, especially of collagen-comprising tissues, may occur in a tendon or ligament in connection with many different pathological conditions and with surgical or cosmetic procedures. However, the mechanism and control of the degradation of the fibrous connective tissues is still poorly understood. Some degree of degradation appears to be part of the healing process, but the trigger for such is not known. However, the involvement of MMPs in degradation of tissues and, for example, the creation of scar tissue, and the utility of MMP inhibitors according to the present invention in the inhibition, i.e. prevention, restriction and hindering, of degradation has been confirmed by experimental data.

Referring now to Table 1 below, matrix metalloproteinase's (MMPs) are a family of Zn⁻⁺ dependent neutral metallo-endopeptidases. Phylogenetically they are from the Matrixin subfamily of Family M10 of the MB clan of Metallopeptidases that have HEXGHXXGXXHS Zinc-Binding Motifs. Two conserved histidine residues and a glutamate immobilise the zinc ion at the active site. At least eleven different types of MMP are known and are designated MMP 1-13, MT-1 MMP and also PUMP 1.

MMPs are secreted in an inactive zymogen form following, cleavage of a signal peptide. They then require further proteolytic cleavage to become activated in the extracellular environment. The activation of MMPs can occur via many mechanisms, these include chaotropic agents (e.g. sodium dodecyl sulphate), low pH, chemicals which can oxidise the sulphydryl group (e.g. N-methyl maleimide) or enzyme proteolysis. In vivo it is likely that the first step of activation is mediated by serine proteinases such as trypsin, plasmin, cathepsin G and kallikrein or other MMPs. These proteases remove part of a 10 kDa propeptide on the N-amino side of a cysteine residue which, in the inactive form is covalently linked by a sulphydryl bond to the zinc atom at the centre of the active region. This bond is consequently de-stabilized and the remainder of the propeptide region is auto catalytically cleaved, producing the active enzyme.

In vitro substrate specificity varies between the different types of MMP although all MMP are able to degrade at least one extra cellular matrix component (e.g. collagen). Different MMPs can lyse the same substrate, although different affinities and kinetics are apparent between the MMPs for a particular substrate. Each MMP can also lyse a variety of substrates although some substrate preference is apparent (see Table 1). Binding of MMP to substrate is site specific, the MMP binding to a particular part of the substrate molecule, and involves the N or the C terminal of the enzyme, which is a specific MMP dependent event. With the exception of MMP-8 and MMP-9 in Neutrophils, MMPs are not found sequestered in storage granules in cells. They are synthesized in response to cell signals and MMP production is controlled at the transcriptional level.

TABLE 1
Matrix Metalloproteinase And In Vitro Substrate Specificity

| | | | MW DA | |
|---|---|---|---|---|
| | | MMP NO./ | PROENZYME/ | |
| ENZYMES | | EC. NO. | ACTIVE | SUBSTRATE |
| 1. | Interstitial | | | |
| | Collagenase Group | | | |
| | Fibroblast Type | MMP-1 | 52000/42000 | I, II, III, VII, |
| | | 3.4.24.7 | | VIII, X |
| | | | | Collagens, Gelatin, |
| | | | | IGFBP-3 |
| | Neutrophil Type | MMP-8 | 58000/57000 | I, II, III Collagens |
| | | 3.4.24.34 | | |
| | Collagenase-III | MMP-13 | 65000/55000 | I, II, III |
| | | | 48000 | Collagens, GelaTin |
| 2. | Gelatinase/Type IV | | | |
| | Collagenase Group | | | |
| | Gelatinase A | MMP-2 | 72000/67000 | I, IV, V, VII, X |
| | | 3.4.24.24 | | Collagens, Gelatin, |
| | | | | Fibronectin, |
| | | | | Elastin |
| | Gelatinase B | MMP-9 | 92000/67000 | IV, V, VII, X |
| | | 3.4.24.35 | | Collagens, Gelatin, |
| | | | | Elastin |
| 3. | Stromelysin Group | | | |
| | Stromelysin-1 | MMP-3 | 57000/45000 | Proteoglycan, |
| | | 3.4.24.17 | 28000 | MMPs-1-9, |
| | | | | Fibronectin, IV, |
| | | | | V, VII, IX, X |
| | | | | Collagens, Laminin, |
| | | | | Gelatin as for |
| | | | | MMP-3 |
| | Stromelysin-2 | MMP-10 | 57000/48000 | |
| | | 3.4.24.22 | 28000 | |
| | Stromelysin-3 | MMP-11 | 58000/28000 | Proteoglycan/ |
| | | | | Fibronectin, Gelatin, |
| | | | | Laminin, |
| | | | | Collagen IV |
| 4. | Others | | | |
| | Matrilysin | MMP-7 | 28000/19000 | Gelatin, Elastin, |
| | (PUMP-1) | 3.4.24.23 | | Fibronectin, |
| | | | | Laminin, Proteoglycans, |
| | | | | Collagen IV, |
| | | | | MMPs1-9 |
| | Macrophage | MMP-12 | 54000/45000 | Elastin, Fibronectin |
| | Metalloelastase | | 22000 | |
| | Membrane Type | MT-MMP | 66000/ | MMP-2 |
| | MMP | | | |

Preferred MMP inhibitors prevent MMP production by a cell. For example, agents may prevent MMP gene transcription, prevent translation of MMP from MMP mRNA, disrupt post-translational modification of MMP, disrupt MMP secretion from the cell in which it is expressed or prevent the formation of active MMP from the zymogen. Alternatively, the inhibitor may be an agent which increases degradation of MMP, such as a proteolytic enzyme. Equally the inhibitor may be an agent which prevents MMP combining with its substrate such as a neutralising antibody against MMP or an aptamer against MMP. The inhibitor may also be an antisense oligonucleotide or ribozyme against MMP mRNA or the MMP gene as appropriate.

Most preferred MMP inhibitors are compounds which selectively inhibit the enzymic action of MMPs by binding to MMP. These may be competitive inhibitors (i.e. those which compete for the active site of MMP) or non-competitive inhibitors (such as allosteric inhibitors or compounds which covalently modify the active site of MMP). Accordingly, the term “MMP inhibitor” is used herein to denote any substance that is capable of inhibiting, i.e. restricting, hindering or preventing, the action of an MMP.

Both natural and synthetic MMP inhibitors are known and may be used according to the present invention. Examples of such naturally occurring MMP inhibitors are α₂-macroglobulin (a collagenase inhibitor found in blood) and Tissue Inhibitors of MMPs (TIMPS).

Preferred synthetic MMP inhibitors include N-[2(R)-2-(hydroxamidocarbonylmethyl)-4-methylpentanoyl]-L-tryptophan methylamide, also known as GM6001, Galardin or Galardin-MPI (trade names), and Batimastat (BB-94). U.S. Pat. No. 5,183,900, U.S. Pat. No. 5,189,178 and U.S. Pat. No. 5,114,953, and EP-A-276436 describe the synthesis of these and other MMP inhibitors, and may all be used according to the present invention. Thus, the inhibitors disclosed therein are incorporated herein by reference.

Most preferred synthetic MMP inhibitors are ganic molecule based on hydroxamic acid. For instance, the inhibitors based on hydroxamic acid, such as [4-(N-hydroxyamino)-2R-isobutyl-3S-(thio-phenyl-thiomethyl)succinyl]-L-phenylalanine-N-methylamide (especially good) and [4-(N-hydroxyamino)-2R-isobutyl-3S-(thiomethyl)succinyl]-L-phenylalanine-N-methylamide and [4-(N-hydroxyamino)-2R-isobutylsuccinyl]-L-phenylalanine-N-(3-aminomethylpyridine)amide and [4-N-hydroxyamino)-2R-isobutyl-3S-methylsuccinyl]-L-phenylalanine-N-[4-(2-aminoethyl)-morpholino]amide are disclosed in WO 90/05716, WO 90/05719, WO 92/13831, and may all be used according to the present invention and the inhibitors disclosed therein are incorporated herein by reference. Further MMP inhibitors are also well known to the art. EP-A-126,974, EP-A-159,396, U.S. Pat. Nos. 4,599,361 and 4,743,587 may all be used according to the present invention and the inhibitors disclosed therein are incorporated herein by reference.

An important form of regulation of MMPs in the ECM is through the activity of specific inhibitors of MMPs known as TIMPs. At least three types of vertebrate TIMP are known (TIMP-1, 2 and 3). The family of TIMPs are defined by a highly conserved secondary structure involving six disulphide bonds. TIMPs bind to MMPs with a 1:1 stoichiometry and inactivate the enzyme. They are frequently produced by the same cell producing MMPs and as the TIMP-MMP binding is tight, production of equimolar concentrations would not lead to an effect on ECM degradation. Thus subtle perturbations in extra-cellular concentrations of either could have a significant impact on ECM degradation.

All TIMPs bind all active MMPs and inhibit them although with varying affinities. Both the N and C terminal domains appear to be important in TIMPs for binding, although the binding of TIMPs to active MMPs seems to involve a variety of mechanisms. TIMP-2 binds pro-MMP-2 and TIMP-1 binds pro-MMP-9. Both these enzymes are capable of subsequent activation, albeit at a lower level and the presence of TIMPs appear to stabilize them against subsequent loss of activity through further cleavage. TIMP-3, like TIMPs 1 and 2, has growth factor like properties, extensive intra-chain disulphide bonding, a molecular weight of 24 kDa and is synthesized in fibroblasts. TIMPs concentration in the ECM is probably regulated through their susceptibility to proteolysis by serine proteases. This mechanism may control local concentration in the ECM.

Developments in TIMP research suggest that TIMP-1 and TIMP-2 are multifunctional proteins with diverse actions. Both inhibitors exhibit growth factor-like activity and can inhibit angiogenesis. TIMP-1 is inducible, glycosylated and X-linked whereas TIMP-2 appears to be constitutively expressed, non-glycosylated and autosomal. TIMP-1 like many of the MMPs has an AP-1 promoter region upstream. This may explain the co-ordinate expression of MMPs and TIMP-1. TGF-β, retinoic acid and female sex hormones, however, up-regulate TIMP-1 and down-regulate the expression of some MMPs. TGF-β specifically up-regulates MMP-2 and 9 and TIMP-1 down-regulates MMP-1 and 3. This contrasts with the actions of IL-4 which down-regulates both MMP-1 and 2 and has no effect on TIMP, which further contrasts with FGF-2 which has an effect through up-regulating MMP-1 and TIMP-1. This would enable complex cocktails of growth factors to have very subtle influences on tissue degradation.

Biomembrane Sealing Agents

For more than 40 years, biomembrane sealing agents of various molecular weights have been utilized as adjuncts to culture media for their ability to protect cells against fluid-mechanical injuries. These agents include hydrophilic polymers such as polyoxyethylenes, PEG, polyvinyl alcohol, amphipatic polymers such as pluronics or poloxamers, including poloxamer P-188 (also known as CRL-5861, available from CytRx Corp., Los Angeles, Calif.) (Michaels and Papoutsakis, 1991) as well as methyl cellulose (Kuchler et al., 1960), sodium carboxylmethyl cellulose, hydroxyethyl starch, polyvinyl pyrrolidine and dextrans (Mizrahi and Moore, 1970; Mizrahi, 1975; Mizrahi, 1983).

Some biomembrane sealing agents including hydroxyethyl starch (Badet et al., 2005) and PEG (Faure et al, 2002; Hauet et al., 2001) have shown effective cryopreservative abilities in organ transplantation studies. Poloxamer P-188 was shown to protect articular cells from secondary injury following mechanical trauma to knee joint which could lead to acute pain and inflammation and potentially develop into a more chronic condition known as osteoarthritis (Phillips and Haut, 2004). Poloxamer P-188 and a neutral dextran protected muscle cells against electroporation or thermally driven cell membrane permeabilization (Lee et al., 1992). Direct application of PEG was shown to anatomically and functionally reconnect transected or crushed axon (Bittner et al., 1986), peripheral nerve (Donaldson et al., 2002) and spinal cord preparations in vitro (Lore et al., 1999; Shi et al., 1999; Shi and Borgens, 1999; Shi and Borgens, 2000; Luo et al., 2002) or in vivo (Borgens et al., 2002). Intravenous or subcutaneous administration of PEG or Poloxamer P-188 improved the cutaneous trunchi muscle reflex response after experimental spinal cord contusion in guinea pigs (Borgens and Bohnert, 2001; Borgens et al., 2004) and improved functional recovery in a naturally occurring spinal cord injury model in dogs (Laverty et al., 2004). PEGs of various molecular weights from 1,400-20000 Da, having a linear or multiple arms structure were shown to improve recovery following tissue injury (Hauet et al., 2001; Detloff et al., 2005; Shi et al., 1999).

Biomembrane sealing agents can be effective following different modes of delivery including local and prolonged cellular exposure, direct and short-term tissue or organ exposure or systemic administration. Effective concentrations of biomembrane fusion agents may vary depending on the purpose and/or mode of delivery For example, about 0.05% concentration is effective in tissue culture applications (Michaels and Papoutsakis, 1991) and about 30% to about 50% concentration is effective for organ preservation and upon in vivo administration in animals (Hauet et al., 2001; Shi et al., 1999; Borgens and Bohnert, 2001; Borgens et al., 2004).

One aspect of the present invention contemplates a combined treatment with PEG and TIMPs to control the activity of the MMPs in the ECN in order to treat and prevent extracellular matrix degradation in an injured tendon or ligament. For example, Applicant and other co-inventors have recently disclosed in co-pending patent application entitled “Compositions Comprising Biomembrane Sealing Agent For Treatment Of Pain Or Inflammation, And Methods Of Use” filed on May 3, 2006, a synergistic effect between PEG, a biomembrane sealing agent, and magnesium is highly significant as it can lead to the development of therapeutic formulations with improved efficacy for the treatment of neuronal trauma, inflammatory and painful conditions. These results suggest that a biomembrane sealing agent, such as, for example, PEG, may also potentiate the beneficial effects of other therapeutic agents including TIMPs.

Referring now to FIG. 2, delivery of the TIMPs may be accomplished in any number of methods. A preferable method begins in step 10 with selection of a form of the medicament suitable for administration to an injured tendon or ligament. Indeed, the medicaments of the invention may take a number of different forms depending, in particular on the manner in which the composition is to be used. Thus, for example, the medicament may be in the form of a powder, tablet, capsule, liquid, ointment, cream, gel, hydrogel, aerosol, spray, micelle, liposome or any other suitable form that may be administered to a person or animal. In step 20, an MMP inhibitor is incorporated into the medicament. Thus, in general the medicaments will usually comprise at least an MMP inhibitor and a pharmaceutically acceptable vehicle. In step 30, the medicament is administered to the injured tendon or ligament. Accordingly, it will be appreciated that the vehicle should be one which is well tolerated by the subject to whom it is given and enables delivery of the MMP inhibitor to the target connective tissue. The vehicle is ideally biocompatible, biodegradable, bioresorbable and non-inflammatory.

The medicament may be used in a number of ways. For instance, a preferred means of administration of a MMP inhibitor for the prevention or reduction of degradation of connective tissue is by topical application. In this case liposomes, micelles, creams, ointments, gels and liquids may be used. A medicament according to the invention (in the form of an ointment or cream for example) may be applied to a tendon through an open wound (which may be an accidental injury or arise from elective surgery). Alternatively a MMP inhibitor may be incorporated in a micelle or liposome and delivered as a spray or aerosol to the target tissue.

Oral formulations may also be used. These may be in the form of tablets, capsules, powders, granules, lozenges or liquid or gel preparations. Tablets may be coated by methods well known in normal pharmaceutical practice. Liquid formulations include syrups. Oral formulations may be used to treat directly conditions such as stomach ulcers and may also be used to treat conditions systemically.

The inhibitor(s) may be dissolved or dispersed in a diluent or carrier. The choice of carrier depends on the nature of the inhibitor, its solubility and other physical properties, and on the method and site of application. For example, only certain carriers are suitable for preparations for use in the eye. Carriers include ethylene glycol, silver sulphadiazine cream and hypromellose. These may be used in creams and drops. An acetate buffer system may also be used.

Further pharmaceutically suitable materials that may be incorporated in pharmaceutical preparations include absorption enhancers, pH regulators and buffers, osmolarity adjusters, emollients, dispersing agents, wetting agents, surfactants, thickeners, opacifiers, preservatives, stabilizers and antioxidants, foaming agents and flocculants, lubricants, colourants and fragrances (generally only in primarily cosmetic preparations).

Gels and liposomes may be the preferred delivery method when the inhibitor is an antisense molecule.

Preferably a medicament according to the present invention is applied directly to an open wound or is injected directly into the site of tissue contraction. Suitable medicaments may, however, be applied to the skin surface where the tissue to be treated is below that surface, the active ingredient then being absorbed by and passing through the skin. Penetration enhancers are preferably incorporated in such medicaments.

Medicaments according to the present invention comprising MMP inhibitors, for example, collagenase inhibitors, for use in the inhibition of the degradation of tissues comprising extracellular matrix components, for example, collagen-comprising tissues, may contain further pharmaceutically active ingredients, for example antibiotics, antifungals, steroids, and further enzyme inhibitors, for example, serine protease inhibitors. Further components for certain indications include growth or healing promoters such as epidermal growth factor (EGF), fibronectin and aprotinin. As mentioned above cytokine inhibitors may also be included.

The inhibitors will generally be used in liquid and other non-solid formulations having concentrations of around 0.3 to 500 μg/ml. In some cases, however, higher concentrations may be required. The total amount used and the dose administered will depend on the severity and area of the degradation, the condition causing it and the physical characteristics of the patient and the site and method of administration.

As can be seen, the present invention provides for the use and delivery of Matrix Metalloproteinase (MMP) inhibitors to treat and prevent such extracellular matrix degradation in an injured tendon or ligament in order to promote natural healing and faster recovery.

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

1. A method of making a medicament for treating an injured tendon or ligament of a human or an animal, comprising:
selecting a form of the medicament suitable for administering to the injured tendon or ligament; and
incorporating a Tissue Inhibitor of MMPs (TIMP) in the medicament.

2. The method according to claim 1, wherein the MMP inhibitor is one of a naturally occurring and a synthetically created inhibitor.

3. The method according to claim 2, wherein the naturally occurring MMP inhibitor is one of a α₂-macroglobulin and a Tissue Inhibitor of MMPs (TIMPS).

4. The method according to claim 2, wherein the synthetically created MMP inhibitor is a ganic molecule based on hydroxamic acid.

5. The method according to claim 1, wherein the concentration of the TIMP is 0.3 to 500 μg/ml.

6. The method according to claim 1, wherein the form of the medicament is one of a powder, tablet, capsule, granule, lozenge, liquid, syrup, ointment, cream, gel, hydrogel, aerosol, spray, drops, micelle, and liposome.

7. The method according to claim 1, wherein the medicament is at least one of biocompatible, biodegradable, bioresorbable and non-inflammatory.

8. The method according to claim 1, wherein the TIMP is dissolved or dispersed in the medicament.

9. The method according to claim 1, further comprising:
incorporating an additional pharmaceutically active ingredient in the medicament.

10. The method according to claim 9, wherein the additional pharmaceutically active ingredient is at least one of an antibiotic, antifungal, steroid and further enzyme inhibitor.

11. The method according to claim 9, wherein the additional pharmaceutically active ingredient is at least one of an epidermal growth factor (EGF), fibronectin and aprotinin.

12. The method according to claim 9, wherein the additional pharmaceutically active ingredient is at least one of anabsorption enhancers, pH regulators or buffers, osmolarity adjusters, emollients, dispersing agents, wetting agents, surfactants, thickeners, opacifiers, preservatives, stabilizers or antioxidants, foaming agents or flocculants, lubricants, colourants and fragrances.

13. A method of treating an injured tendon or ligament of a human or an animal with a medicament, comprising:
selecting a form of the medicament suitable for administering to the injured tendon or ligament; and
administering the medicament to the injured tendon or ligament,
wherein the medicament has incorporated a TIMP.

14. The method according to claim 13, wherein administration of the medicament to the injured tendon or ligament is in a therapeutically effective amount.

15. The method according to claim 13, wherein the MMP inhibitor is one of a naturally occurring and a synthetically created inhibitor.

16. The method according to claim 15, wherein the naturally occurring MMP inhibitor is one of a α₂-macroglobulin and a Tissue Inhibitor of MMPs (TIMPS).

17. The method according to claim 15, wherein the synthetically created MMP inhibitor is a ganic molecule based on hydroxamic acid.

18. The method according to claim 13, wherein the concentration of the TIMP is 0.3 to 500 μg/ml.

19. The method according to claim 13, wherein the form of the medicament is one of a powder, tablet, capsule, granule, lozenge, liquid, syrup, ointment, cream, gel, hydrogel, aerosol, spray, drops, micelle, and liposome.

20. The method according to claim 13, wherein the medicament is at least one of biocompatible, biodegradable, bioresorbable and non-inflammatory.

21. The method according to claim 13, wherein the TIMP is dissolved or dispersed in the medicament.

22. The method according to claim 13, wherein an additional pharmaceutically active ingredient is incorporated in the medicament.

23. The method according to claim 22, wherein the additional pharmaceutically active ingredient is at least one of an antibiotic, antifungal, steroid and further enzyme inhibitor.

24. The method according to claim 22, wherein the additional pharmaceutically active ingredient is at least one of an epidermal growth factor (EGF), fibronectin and aprotinin.

25. The method according to claim 22, wherein the additional pharmaceutically active ingredient is at least one of absorption enhancers, pH regulators or buffers, osmolarity adjusters, emollients, dispersing agents, wetting agents, surfactants, thickeners, opacifiers, preservatives, stabilizers or antioxidants, foaming agents or flocculants, lubricants, colourants and fragrances.

26. The method according to claim 13, wherein the medicament further has incorporated a therapeutically effective amount of at least one biomembrane sealing agent.

27. The method according to claim 26, wherein the biomembrane sealing agent is PEG.
